(19) United States
(12) Patent Application Publication
(10) Pub. No.: US 2026/0048127 A1
(43) Pub. Date: Feb. 19, 2026

(54) ULTRASOUND-ASSISTED DELIVERY OF DRUG-LOADED CHITOSAN NANOPARTICLES

(71) Applicant: NATIONAL TAIWAN UNIVERSITY, Taipei (TW)

(72) Inventors: Wei-Wen LIU, Taipei City (TW); Bor-Shiunn LEE, Taipei City (TW); Tzu-Yun HUANG, Taipei City (TW); Qiao-Zhen CHANG, Taipei City (TW)

(21) Appl. No.: 19/300,102

(22) Filed: Aug. 14, 2025

Related U.S. Application Data
(60) Provisional application No. 63/682,906, filed on Aug. 14, 2024.

(51) Int. Cl. A61K 41/00; A61K 9/00; A61K 9/51; A61K 33/243; A61M 37/00

(52) U.S. Cl. CPC A61K 41/0047; A61K 9/0053; A61K 9/5161; A61K 33/243; A61M 37/0092

(57) ABSTRACT

The present invention, based on a non-bubble-based sonoporation technique, provides an ultrasound-assisted delivery platform for drug-loaded chitosan nanoparticles. This platform comprises: the administration of drug-loaded chitosan nanoparticles and the application of ultrasound stimulation. The drug-loaded chitosan nanoparticles are delivered to a target site, where ultrasound waves are directly applied to induce cellular membrane deformation. The ultrasound mechanical force temporarily alters membrane permeability, thereby facilitating the intracellular delivery of the drug-loaded chitosan nanoparticles.

### CROSS-REFERENCE TO RELATED APPLICATIONS

This application claims the benefit of priority under 35 U.S.C. § 119 (e) to U.S. Provisional Patent Application No. 63/682,906, filed Aug. 14, 2024. The above-mentioned application is hereby incorporated by reference herein in its entirety.

### FIELD OF THE INVENTION

This disclosure relates to the field of drug delivery, and more specifically to an ultrasound-assisted chitosan nanoparticle drug delivery platform that utilizes a non-bubble-based sonoporation technique.

### BACKGROUND OF THE INVENTION

Chemotherapy is a therapeutic approach that employs chemical agents to inhibit or destroy cancer cells, thereby suppressing cancer progression. Its primary mechanism involves interfering with cellular proliferation by damaging DNA or RNA, which prevents cell division and repair, ultimately leading to tumor control or shrinkage. A distinctive feature of chemotherapy is its systemic nature, meaning that the administered drugs circulate through the bloodstream and reach various body parts. Owing to this property, chemotherapy has become one of the primary treatment modalities for metastatic cancers. Chemotherapy encompasses a broad spectrum of drugs with diverse mechanisms of action, such as alkaloids and antimetabolites that disrupt cell division and interfere with DNA or RNA synthesis to inhibit cell proliferation, as well as antitumor agents that bind to DNA and prevent its replication, ultimately causing cell death. However, because most chemotherapeutic agents lack specificity toward cancer cells, they often affect normal cells, particularly those with high proliferation rates, such as hair follicle cells, bone marrow cells, and gastrointestinal mucosal cells. This non-selectivity results in a range of systemic side effects, including leukopenia, anemia, and thrombocytopenia due to bone marrow suppression (increasing the risk of infection and anemia), nausea, vomiting, and diarrhea due to gastrointestinal mucosal damage, and alopecia caused by injury to hair follicle cells. These adverse effects significantly exacerbate patient suffering. Mitigating the systemic side effects of chemotherapy is thus crucial for improving its clinical utility. With recent advances in medicine, the emergence of targeted therapies, immunotherapies, and advanced drug delivery systems has reduced collateral damage to healthy cells, thereby enhancing treatment precision and therapeutic outcomes.

Cisplatin, also known as cis-diamminedichloroplatinum (II) (CDDP), is a widely used chemotherapeutic agent and a platinum-based anticancer drug. Its primary mechanism of action involves forming crosslinks with intracellular DNA, thereby disrupting the DNA repair machinery of cancer cells, inducing DNA damage, and ultimately triggering apoptosis. Cisplatin has demonstrated significant therapeutic efficacy across multiple malignancies, including testicular cancer, ovarian cancer, head and neck cancer, and lung cancer. Pharmacologically, when administered intravenously, cisplatin remains stable in the bloodstream because of its relatively high chloride concentration (˜100 mM), which preserves the integrity of its chloride ligands bound to the Pt (II) center. Upon entry into the intracellular environment, where the chloride concentration is significantly lower (˜4 mM), these chloride ligands are replaced by water molecules, forming positively charged aquated complexes. These reactive species readily bind to nucleophilic sites on cellular macromolecules such as proteins, RNA, and DNA. In DNA specifically, cisplatin primarily forms intrastrand crosslinks at the N7 positions of guanine and adenine, along with a smaller proportion of interstrand crosslinks and monofunctional adducts. These DNA adducts disrupt DNA replication and RNA transcription, leading to cell cycle arrest at the G2 phase and, ultimately, programmed cell death (apoptosis).

The administration of chemotherapeutic agents faces numerous challenges, including toxicity to normal cells, poor bioavailability, and insufficient stability. In recent years, advances in nanotechnology have provided new insights and solutions for drug delivery. The use of nanoparticles as carriers for anticancer agents has emerged as a promising chemotherapy strategy. Due to their controlled drug release profiles and tumor-targeting capabilities, nanoparticles represent ideal delivery vehicles for anticancer therapeutics. Their design and functional properties are highly dependent on the type of drug encapsulated. By improving drug stability and enabling controlled release, nanoparticles enhance intratumoral drug accumulation while reducing toxicity and damage to normal cells, thereby improving therapeutic efficacy and minimizing adverse effects. Common nanoparticle-based carriers include liposomes, solid lipid nanoparticles, polymeric nanoparticles, dendrimers, and gold nanoparticles. Among these, liposomal nanoparticles were the first reported and subsequently approved by the FDA in 1995, and such carriers are now widely applied in clinical settings. The endocytic uptake of nanoparticles largely relies on physicochemical interactions between nanoparticles and cells, including electrostatic forces, van der Waals forces, hydrophobic interactions, and ligand-receptor binding. Critical factors such as nanoparticle size, shape, and surface modification influence these interactions and determine the efficiency of cellular internalization. Particle size, in particular, dictates the cellular entry pathway: micron-sized particles are typically internalized through phagocytosis or macropinocytosis, whereas nanoscale particles primarily utilize clathrin-dependent and caveolae-dependent endocytic pathways. The internalization of nanoparticles is a complex process influenced by multiple parameters, including particle size, morphology, surface characteristics, and receptor diffusion kinetics.

Chitosan, also known as chitin-glucan, is a linear polysaccharide derived from chitin, a natural polymer obtained primarily from the exoskeletons of crustaceans. As a biomaterial characterized by excellent biocompatibility, high biodegradability, antimicrobial properties, antioxidant activity, and low cytotoxicity, chitosan has demonstrated broad application potential across diverse fields, including biomedicine, drug delivery, and industry. Owing to its favorable biocompatibility and antimicrobial nature, chitosan has been widely used in medical applications, such as the fabrication of wound dressings. In biomedical engineering, chitosan serves as a scaffold material to mimic the structure of the extracellular matrix, thereby facilitating cell adhesion and growth. Within drug delivery systems, its biocompatibility and biodegradability make chitosan an especially attractive carrier material. The amino and hydroxyl functional groups in its molecular structure allow various chemical modifications to enhance solubility and drug-loading capacity. Moreover, such chemical modifications enable fine control over drug release kinetics-for example, conjugation with polyethylene glycol (PEG) improves aqueous stability and modulates drug release profiles.

Ultrasound is a form of physical mechanical energy with significant applications across multiple disciplines, including medicine and engineering. In the medical field, ultrasound is widely regarded as a convenient, safe, and effective imaging modality. Initially used for anatomical imaging and diagnostic purposes, ultrasound, when combined with molecular imaging and microbubble contrast agents, enables the targeting of specific disease markers, including cancer and other pathological lesions, thereby facilitating high-sensitivity visualization of molecular activities. Its high spatial resolution further enables micron-scale vascular imaging, substantially improving the accuracy and success rate of medical diagnostics and interventions. Recent advances in technology and nanomedicine have greatly expanded the use of ultrasound in molecular imaging and drug delivery. In drug delivery, ultrasound plays a critical role as a facilitator of therapeutic transport. Numerous studies have reported its effectiveness in enhancing drug delivery, including applications such as opening the blood-brain barrier (BBB) to treat brain tumors and neurodegenerative disorders. This approach, particularly when combined with microbubbles, increases cell membrane permeability and facilitates drug penetration across biological barriers.

Microbubbles are micron-sized gas-filled bubbles typically encapsulated by a lipid shell. The integration of ultrasound technology with microbubbles has emerged as an effective method for enhancing drug delivery through sonoporation. This technique has been widely applied in various medical fields. In cancer therapy, due to the chaotic and leaky architecture of tumor vasculature, ultrasound-microbubble-mediated delivery can effectively enhance drug penetration into tumor tissues, thereby reducing systemic toxicity and improving therapeutic efficacy. When exposed to ultrasound, microbubbles oscillate, undergoing continuous expansion and contraction. When microbubbles are exposed to ultrasound, they oscillate, resulting in continuous expansion and contraction. This generates a mechanical force that causes temporary pores in the cell membrane, increasing its permeability. This allows drugs to enter cells or other biological barriers. Additionally, the energy from ultrasound may cause the microbubbles to rupture, enabling precise release of the loaded drugs at the target site. Despite its remarkable potential, the safety of microbubble-mediated sonoporation remains a critical concern. Literature reports indicate that bubble-based sonoporation can significantly impact cell viability. Although higher peak negative pressures may improve delivery efficiency, they often cause a substantial decrease in cell viability. Key acoustic parameters-such as ultrasound pressure, frequency, and exposure duration-profoundly influence microbubble dynamics, thereby affecting both the mechanism and overall performance of sonoporation as well as its biological consequences. Although the combination of ultrasound and microbubbles for acoustic perforation shows great potential and is currently the mainstream approach, it also faces challenges such as loss of cell viability and other performance imbalances, which are the predicaments of bubble-based acoustic perforation.

In recent years, non-bubble-based sonoporation has been proposed as a novel approach to enhance cell membrane permeability without the use of microbubbles. This refers to a method that uses acoustic waves to act on cell membranes without the use of microbubbles, temporarily opening pores in the cell membranes to increase their permeability. Compared to bubble-based sonoporation, this technology does not rely on the formation and rupture of bubbles to create pores. Instead, it utilizes the acoustic radiation force and shear force generated by ultrasound propagating through a fluid to achieve the same effect. By directly applying these acoustic forces to the cell membrane, localized membrane deformation is induced, resulting in temporary changes in membrane permeability that facilitate the intracellular delivery of therapeutic agents. Because this approach does not depend on shear forces generated by microbubbles, it eliminates the need to control parameters such as bubble concentration and proximity, enabling more precise and efficient intracellular drug delivery while reducing adverse effects on cell viability commonly associated with microbubble-induced sonoporation.

In summary, the development of platform technologies capable of delivering chemotherapeutic agents with precision, high efficiency, and robust efficacy will represent a critical medical need in the future.

### SUMMARY OF THE INVENTION

The present invention, based on non-bubble-based sonoporation, provides an ultrasound-assisted drug-loaded chitosan nanoparticle delivery platform comprising: supplying drug-loaded chitosan nanoparticles; providing an ultrasound stimulation; administering the drug-loaded chitosan nanoparticles to a target region; and applying the ultrasound stimulation to the target region using either a low-frequency non-focused or focused probe, and wherein the ultrasound stimulation induces localized deformation of cell membranes, resulting in transient increases in membrane permeability that enable the intracellular delivery of the drug.

In some embodiments, the target region is oral.

In some embodiments, the operating time provided by the low-frequency non-focused or focused probe is at a duty cycle of 50% and within a range of 50 to 300 ms.

In some embodiments, the duration of the ultrasonic stimulation is between 50 and 90 seconds.

In a preferred embodiment, the duration of ultrasonic stimulation is 60 seconds.

In some embodiments, the drug-loaded chitosan nanoparticles are loaded with cisplatin.

In an embodiment, the drug-loaded chitosan nanoparticles are provided in a drug patch.

In some embodiments, the particle size of the drug-loaded chitosan nanoparticles is 300 to 500 nm.

In some embodiments, the drug-loaded chitosan nanoparticles with a 300 to 400 nm particle size provides optimal therapeutic efficacy.

### BRIEF DESCRIPTION OF THE DRAWINGS

FIG. 1 shows the particle size analysis of cisplatin-loaded chitosan nanoparticles prepared at a chitosan/Tripolyphosphate (TPP) mass ratio of 15:1 (n=3)

FIG. 2 shows the calibration curve of cisplatin in double-distilled water (ddH₂O)

FIG. 3 shows the calibration curve of cisplatin in phosphate buffered saline (PBS).

FIG. 4 shows the release profile of cisplatin from cisplatin-loaded chitosan nanoparticles at a chitosan/TPP mass ratio of 15:1 (n=4).

FIG. 5 shows that the Percentage release profile of cisplatin from cisplatin-loaded chitosan nanoparticles at a chitosan/TPP mass ratio of 15:1 (n=4)

FIG. 6 shows the schematic illustration of ultrasound-assisted drug delivery experiments: (A) structural diagram of the vertical glass chamber; (B) schematic representation of mouse skin and drug patch placement within the vertical glass chamber.

FIG. 7 shows that the effect of ultrasound on the release of cisplatin-loaded chitosan nanoparticles (*P<0.05 and **P<0.01)

FIG. 8 shows that the effect of cisplatin-loaded chitosan nanoparticles release on MOC2cell viability (*P<0.05, **P<0.01, and ***P<0.001)

FIG. 9 shows that the effect of ultrasound-triggered release from cisplatin-loaded chitosan nanoparticles on MOC2 cell viability (*P<0.05, **P<0.01, and ***P<0.001)

FIG. 10 shows the sonoporation effect induced by ultrasound in MOC2 cells. Scale bar=100 82 m

FIG. 11 shows the percentage of sonoporation induced by ultrasound in MOC2 cells (**P<0.001)

FIG. 12 shows the ultrasound B-mode images of untreated (NTX) tumors in axial and longitudinal orientations.

FIG. 13 shows the ultrasound B-mode images of tumors treated with agarose drug patch passive diffusion (aCisNP) in axial and longitudinal orientations.

FIG. 14 shows the ultrasound B-mode images of tumors (no drug patch) subjected to ultrasound exposure for 60 seconds with a working time of 70 ms (US 60 s/70 ms) in axial and longitudinal orientations.

FIG. 15 shows the ultrasound B-mode images of tumors (no drug patch) subjected to ultrasound exposure for 60 seconds with a working time of 250 ms (US 60 s/250 ms) in axial and longitudinal orientations.

FIG. 16 shows the ultrasound B-mode images of tumors treated with drug delivery using ultrasound exposure for 60 seconds with a working time of 70 ms (US 60 s/70 ms+aCisNP) in axial and longitudinal orientations.

FIG. 17 shows the ultrasound B-mode images of tumors treated with drug delivery using ultrasound exposure for 60 seconds with a working time of 250 ms (US 60 s/250 ms+aCisNP) in axial and longitudinal orientations.

FIG. 18 shows that the bar chart of tumor volume changes in each group measured by a high-frequency ultrasound imaging system (*P<0.05)

FIG. 19 shows that the platinum content in tumor tissues measured by ICP-MS (*P<0.05)

### DETAILED DESCRIPTION OF THE INVENTION

### Definition

The ultrasound-assisted chitosan nanoparticle drug delivery platform of the present invention utilizes chitosan nanoparticles capable of encapsulating any drug that can be incorporated within such nanoparticles, including, but not limited to, conventional pharmaceuticals, chemotherapeutic agents, targeted therapeutics, and anticancer drugs.

To demonstrate the feasibility of the ultrasound-assisted chitosan nanoparticle drug delivery platform of the present invention, subsequent experimental embodiments employ chitosan nanoparticles loaded with the chemotherapeutic agent cisplatin as an illustrative example. However, this embodiment is provided solely for reference and is not intended to limit the platform to cisplatin-loaded nanoparticles.

### EXAMPLE 1

### Preparation of Cisplatin-loaded Chitosan Nanoparticles

Preparation method for cisplatin-loaded chitosan nanoparticles (prepared at a chitosan-to-Tripolyphosphate (TPP) mass ratio of 15:1): Chitosan powder (medium molecular weight, Sigma-Aldrich, USA) solution was dissolved in 40 mL of 1% acetic acid solution and homogenized using ultrasonic energy at approximately 33 W for 3-8 minutes. Following ultrasonic treatment, 4 mL of 1.25 mg/mL cisplatin solution was added and stirred for 1-3 minutes, after which 200 μL of Tween 80 was introduced and stirred for 3-8 minutes. The mixture was homogenized using ultrasonic energy at approximately 33 W for 5-10 minutes. The pH of the resulting solution was adjusted to 4.5-5 using 1 N NaOH, and the TPP solution was slowly titrated into the mixture using a burette. After titration, the solution was stirred for 25-35 minutes to allow crosslinking between chitosan and TPP, forming cisplatin-loaded chitosan nanoparticles. The nanoparticles were subsequently collected by centrifugation at 25-35 minutes to precipitate the nanoparticles.

### Preparation of Agarose Drug Patch

0.06 g of agarose powder was added to 10 mL of double-distilled water (ddH₂O) and heated until fully dissolved. Upon cooling to 55-65° C., 200 μL of the agarose solution was mixed with 150 μg of cisplatin-loaded chitosan nanoparticles, and the mixture was cast in a mold to form a patch measuring 1×1×0.3 cm. The patch was then sterilized using 400 W UV irradiation for 15 minutes, resulting in an agarose drug patch, which was subsequently used in ultrasound-mediated drug delivery experiments.

### EXAMPLE 2

### Particle Size and Zeta Potential Analysis of Cisplatin-loaded Chitosan Nanoparticles

The particle size distribution and zeta potential of cisplatin-loaded chitosan nanoparticles were analyzed using a dynamic light scattering (DLS) analyzer (90 Plus Particle Size Analyzer, Brookhaven, USA) to determine particle size and evaluate surface charge stability. For particle size measurement, 1 mL of the cisplatin-loaded chitosan nanoparticles suspension was placed into a cuvette and analyzed via DLS. For zeta potential measurement, 1.7 mL of the cisplatin-loaded chitosan nanoparticles suspension was loaded into a specialized zeta potential cuvette to measure electrophoretic mobility, thereby confirming the surface charge stability of the nanoparticles.

In this experiment, the particle size and surface charge stability of the previously prepared chitosan/TPP (15:1) cisplatin-loaded chitosan nanoparticles were measured. For particle size determination, 1 mL of the cisplatin-loaded chitosan nanoparticles suspension was placed into a cuvette and analyzed using DLS.

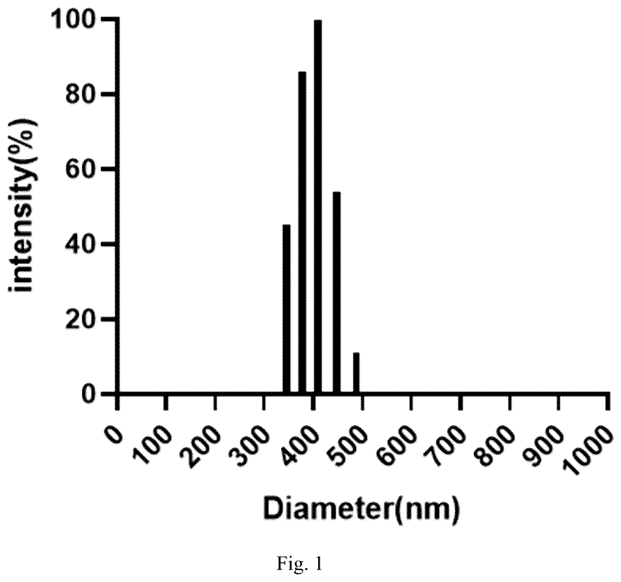

As shown in FIG. 1, the particle size distribution of chitosan/TPP (15:1) cisplatin-loaded chitosan nanoparticles ranged from 345.8 to 487.9 nm.

Subsequently, 1.7 mL of the cisplatin-loaded chitosan nanoparticles suspension was transferred into a specialized zeta potential cuvette, and the electrophoretic mobility of the particles was measured to assess surface charge stability. The results showed that the chitosan/TPP (15:1) cisplatin-loaded chitosan nanoparticles had a surface potential of 35.59 mV, indicating stable surface charge properties.

### EXAMPLE 3

### Encapsulation efficiency of Cisplatin-loaded Chitosan Nanoparticles

### A. Preparation of Cisplatin Calibration Curve

Cisplatin was reacted with o-phenylenediamine (O-PDA) in a dimethylformamide (DMF) solution at 90° C., producing a pale blue product. The reaction product was quantified using UV-Vis spectroscopy at a wavelength of 703 nm. Cisplatin solutions were prepared in either ddH₂O or phosphate buffered saline (PBS) at concentrations of 2 μL/mL, 4 μL/mL, 6 μL/mL, 8 μL/mL, and 10 μL/mL. For each concentration, 0.5 mL of DMF containing 1.4 mg/mL O-PDA was mixed with 0.5 mL of the cisplatin solution and allowed to react at 90° C. until a color change was observed. After cooling, the absorbance was measured at 703 nm by UV-Vis spectroscopy, and a cisplatin/ddH₂O calibration curve was constructed.

### B. Measurement of Drug Encapsulation Efficiency

The supernatant obtained after centrifugation in Example 1 was collected, diluted 10-fold with ddH₂O, and thoroughly mixed with DMF containing 1.4 mg/mL O-PDA. The mixture was reacted at 90° C. until a color change was observed, then cooled and analyzed by UV-Vis spectroscopy at 703 nm. The drug encapsulation efficiency was calculated using the following formula:

Drug Encapsulation Efficiency(%)=[(Initial Drug Concentration-Drug Concentration in Supernatant)+Initial Drug Concentration]×100%.

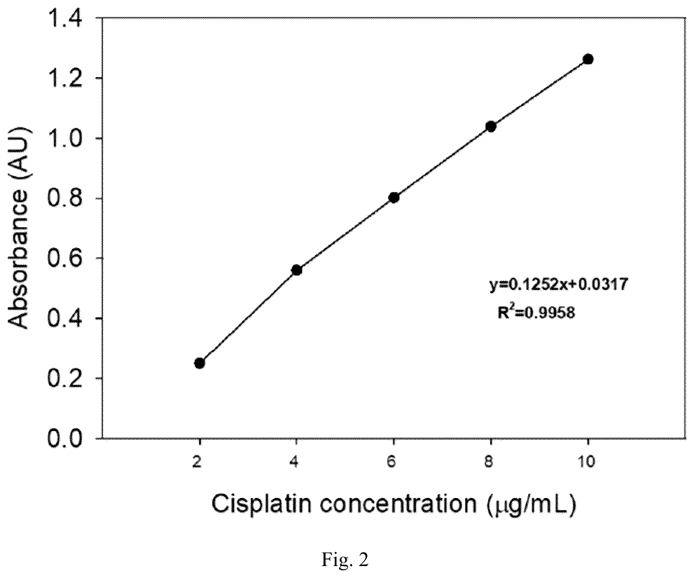

As shown in FIG. 2, the absorbance values obtained were 0.25, 0.56, 0.80, 1.04, and 1.26, with a calibration curve R² value of 0.9958, indicating high reliability. The encapsulation efficiency of the previously prepared cisplatin-loaded chitosan nanoparticles with a chitosan/TPP mass ratio of 15:1 was determined to be 58.57%.

### EXAMPLE 4

### Drug Release Efficiency of Cisplatin-loaded Chitosan Nanoparticles

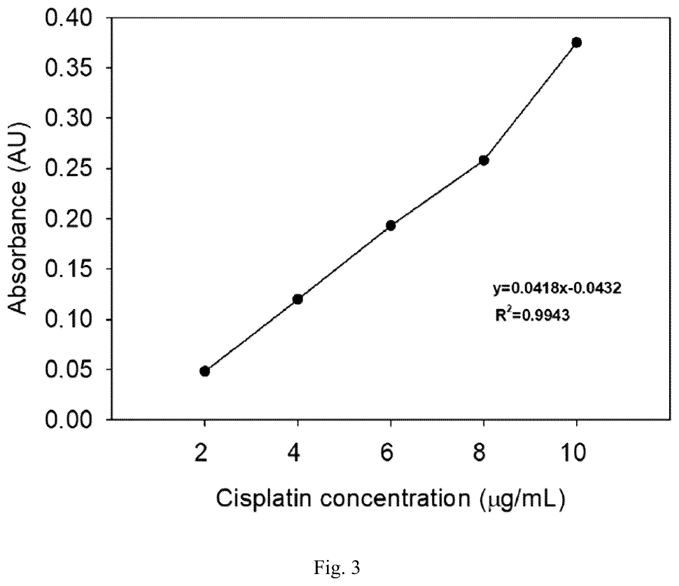

First, the absorbance of cisplatin was measured using the same method described in Example 3 to establish a cisplatin/PBS calibration curve. Cisplatin was prepared in PBS at concentrations of 2, 4, 6, 8, and 10 μL/mL, and the absorbance was measured to generate the calibration curve. As shown in FIG. 3, the measured absorbance values were 0.048, 0.12, 0.193, 0.258, and 0.375, with an R² value of 0.9943, indicating a highly reliable calibration curve.

To evaluate the drug release behavior of the previously prepared cisplatin-loaded chitosan nanoparticles (chitosan/TPP mass ratio 15:1) and confirm their release efficiency, the centrifuged nanoparticle pellet was immersed in 5 mL of 1×PBS and incubated at 37° C. At predetermined time points (0.5, 3, 5, 24, 48, and 72 hours), 700 μL of supernatant was withdrawn and replaced with an equal volume (700 μL) of fresh 1×PBS to maintain a total volume of 5 mL. The concentration of released cisplatin was determined by measuring absorbance at 703 nm using UV-Vis spectroscopy and referencing the previously established calibration curve.

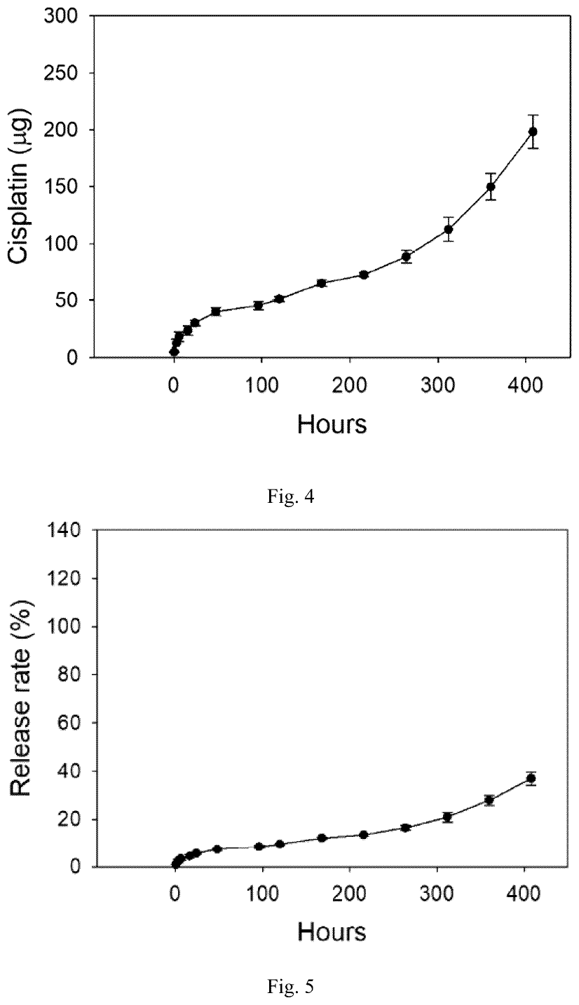

As shown in FIGS. 4-5, cumulative cisplatin release from 0.5 hours to 72 hours was measured at 4.71 μg, 12.35 μg, 18.28 μg, 23.63 μg, 30.09 μg, and 40.03 μg, corresponding to release rates of 0.87%, 2.29%, 3.40%, 4.39%, 5.60%, and 7.45%, respectively.

### EXAMPLE 5

### Ultrasound-Assisted Drug Delivery Experiment Pretreatment

### A. Mouse Skin Preparation

After euthanizing the mice, the mice hair was removed. The dorsal skin was then excised and immersed in PBS. Skin thickness was measured using a digital caliper.

### B. Transdermal Testing

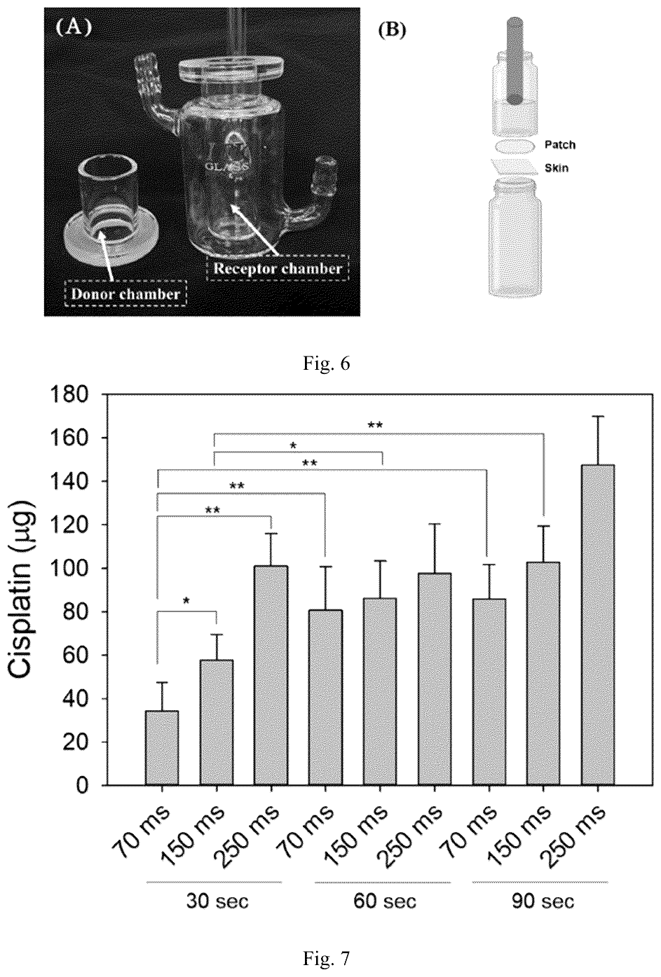

As illustrated in FIG. 6, (A) a custom-made vertical glass chamber with a dual-layer structure was used. (B-C) For each experiment, 12 mL of PBS was added to the lower chamber. A drug patch and mouse skin were positioned at the chamber opening, which was sealed on both sides with Parafilm to ensure tight closure. Subsequently, 4 mL of PBS was added to the upper chamber, and an ultrasound probe was placed in the upper chamber solution to perform negative control, passive diffusion control, and experimental tests under different ultrasound energy parameters.

TABLE 1
Ultrasound Delivery Parameter Table

| Intensity | Time (sec) | Duty cycles (50%) | |
|---|---|---|---|
| 3 W/cm² | 30 | 70 | ms |
| | | 150 | ms |
| | | 250 | ms |
| | 60 | 70 | ms |
| | | 150 | ms |
| | | 250 | ms |
| | 90 | 70 | ms |
| | | 150 | ms |
| | | 250 | ms |

To evaluate the effect of ultrasound parameters on the transdermal delivery of cisplatin-loaded chitosan/TPP nanoparticles, mouse skin samples were randomly assigned to the following groups: negative control, passive release (positive control), and experimental groups with varying total exposure times (30 seconds, 60 seconds, and 90 seconds) and duty cycle settings (50%, 70 ms; 50%, 150 ms; and 50%, 250 ms). Transdermal delivery experiments were conducted using the vertical glass chamber setup described previously (Section B). The solution from the lower chamber was collected to measure the drug release amount and release efficiency.

The results of drug release in the lower chamber solution are shown in FIG. 7. For the 30-second exposure group, the released drug amounts from the agarose patch increased with longer duty cycles, measuring 34.32±13.20 μg, 57.57±12.10 μg, and 101.06±14.97 μg, respectively. For the 60-second exposure group, the released drug amounts were 80.58±20.10 μg, 86.04±17.32 μg, and 97.53±22.74 μg, respectively. For the 90-second exposure group, the released drug amounts were 85.92±15.92 μg, 102.78±16.63 μg, and 147.32±22.35 μg, respectively. These results demonstrate a positive correlation between released drug amount, duty cycle, and total exposure time. At the same total exposure time, longer duty cycles increased cisplatin release. The calculated release rates are presented in Table 2.

TABLE 2
Cisplatin content released into the lower-
phase solution following ultrasound treatment

| | | | Released | Drug release |
|---|---|---|---|---|
| | | | drug (μg) | rate (%) |
| Duty cycle 50% | 70 | ms on/off | 34.32 ± 13.20 | 65.2 ± 25.1 |
| 30 sec | 150 | ms on/off | 57.57 ± 12.10 | 78.3 ± 16.4 |
| | 250 | ms on/off | 101.01 ± 14.97 | 87.5 ± 12.9 |
| Duty cycle 50% | 70 | ms on/off | 80.58 ± 20.10 | 84.8 ± 15.7 |
| 60 sec | 150 | ms on/off | 86.04 ± 17.32 | 85.9 ± 17.2 |
| | 250 | ms on/off | 97.53 ± 22.74 | 87.6 ± 20.4 |
| Duty cycle 50% | 70 | ms on/off | 85.92 ± 15.92 | 87.2 ± 16.6 |
| 90 sec | 150 | ms on/off | 102.78 ± 16.63 | 89.1 ± 18.7 |
| | 250 | ms on/off | 147.33 ± 22.35 | 92.4 ± 20.7 |

### EXAMPLE 7

### Effect of Different Ultrasound Parameters on the Cytotoxicity of Cisplatin-loaded Chitosan Nanoparticles in MOC2 Cells

### A. Effect of Cisplatin-loaded Chitosan Nanoparticles Release on MOC2 Cell Viability in the Absence of Ultrasound

First, to determine whether cisplatin-loaded agarose drug patches (aCisNP) exhibit passive drug release in the absence of ultrasound and whether agarose drug patches without cisplatin exert any cytotoxic effects, patches containing cisplatin (aCisNP) and patches without cisplatin (agarose) were placed on cell culture plates for 90 seconds and then removed. Cell viability was assessed after 24 hours.

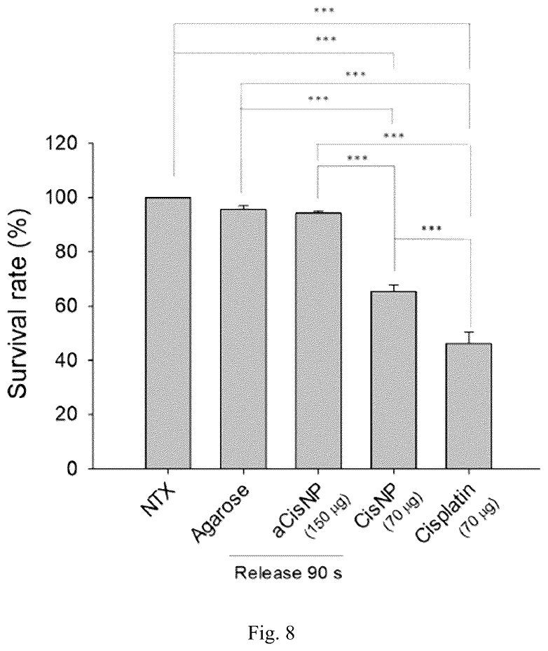

As shown in FIG. 8, in the absence of ultrasound, both the cisplatin-loaded agarose patch (aCisNP) and the unloaded agarose patch exhibited no significant cytotoxicity compared with the control group (NTX) (NTX: 100% vs. agarose: 95.74% and aCisNP: 94.20%). The cytotoxic effects of free cisplatin and cisplatin encapsulated within chitosan/TPP nanoparticles (CisNP) were evaluated. After 24 hours of exposure, cell viability was assessed using the MTT assay. The results showed that free cisplatin reduced cell viability to 46.21% at 24 hours compared to the control group, and CisNP treatment resulted in a cell viability of 65.30%.

### B. Effect of Ultrasound-Mediated Delivery of Cisplatin-loaded Chitosan Nanoparticles on MOC2 Cell Viability

Considering that a total exposure time of 90 seconds may induce excessive cell damage due to high energy input, an additional group with a total exposure time of 75 seconds was included in the experiment. This group was expected to demonstrate a higher drug delivery efficiency than the 60-second group while producing lower cellular damage than the 90-second group. Different total exposure times (30 second, 60 second, 75 second, and 90 second) and duty cycle settings (50%, 70 ms; 50%, 150 ms; and 50%, 250 ms) were tested to evaluate the effect of ultrasound parameters on cisplatin release and subsequent impact on cell viability.

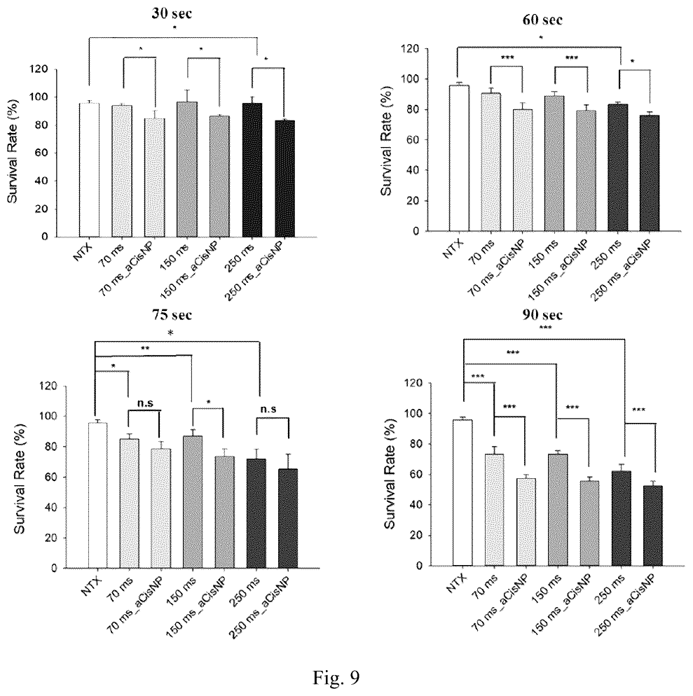

As shown in FIG. 9, compared with the control group (NTX), for a 30-seconds exposure at a duty cycle of 50%, 70 ms, cell viability without aCisNP treatment was 94.05%, showing no significant difference relative to the control (95.66% vs. 94.05%). When aCisNP was applied, cell viability decreased to 84.78%, significantly lower than that in the untreated group (94.05% vs. 84.78%, *P<0.05). At a duty cycle of 50%, 150 ms, cell viability without aCisNP treatment was 91.25%, also showing no significant difference compared with the control (95.66% vs. 91.25%), whereas with aCisNP treatment, cell viability decreased to 78.13%, representing a significant difference relative to untreated cells. At a duty cycle of 50%, 250 ms, cell viability without aCisNP treatment was 86.08%, which was significantly lower than the control (95.66% vs. 86.08%), and decreased further to 76.07% with aCisNP treatment, also showing a significant difference compared with untreated cells (86.08% vs. 76.07%, *P<0.05).

For a 60-seconds exposure, cell viability without aCisNP treatment at duty cycles of 70 ms, 150 ms, and 250 ms was 90.45%, 88.92%, and 83.29%, respectively. Duty cycles of 70 ms and 150 ms showed no significant differences compared with the control (95.66% vs. 90.45% and 88.92%), whereas 250 ms showed a significant reduction (95.66% vs. 83.29%, *P<0.05). With aCisNP treatment, cell viability was further reduced to 80.20%, 79.26%, and 76.07%, significantly lower than their respective untreated groups (*P<0.05).

For a 75-seconds exposure, cell viability without aCisNP treatment at duty cycles of 70 ms, 150 ms, and 250 ms was 84.92%, 86.91%, and 72.06%, respectively, all significantly different from the control (95.66% vs. 84.92%, *P<0.05; 95.66% vs. 86.91%, **P<0.01; 95.66% vs. 72.06%, ***P<0.001). With aCisNP treatment, cell viability decreased to 78.59%, 73.71%, and 65.17%, respectively. Among these, duty cycles of 70 ms and 250 ms showed no significant differences compared with their respective untreated groups (84.92% vs. 78.59%; 72.06% vs. 65.17%), whereas the 150 ms duty cycle group showed a significant difference (86.91% vs. 73.71%, *P<0.05).

For a 90-seconds exposure, cell viability without aCisNP treatment at duty cycles of 70 ms, 150 ms, and 250 ms was 73.21%, 73.37%, and 62.28%, respectively, all significantly lower than the control group (***P<0.001). With aCisNP treatment, cell viability decreased further to 57.42%, 55.69%, and 52.51%, respectively, each showing significant differences compared with untreated groups (73.21% vs. 57.42%; 73.37% vs. 55.69%; 62.28% vs. 52.51%; *P<0.001).

The results demonstrated a negative correlation between duty cycle, total exposure time, and cell viability. At the same total exposure time, longer duty cycles enhanced cellular cytotoxicity and drug release. However, at exposure times of 75 seconds and 90 seconds, the ultrasound energy alone may have induced significant cell death. From a safety perspective, excessive ultrasound-induced cytotoxicity is undesirable; the optimal condition is that ultrasound alone does not significantly differ from the control. However, when combined with drug treatment, it induces pronounced cytotoxicity. Based on these criteria, a 60-seconds exposure time with a 70-ms duty cycle was selected for subsequent animal experiments.

### EXAMPLE 9

### Ultrasound-Induced Sonoporation Effect in MOC2 Cells

To confirm that the selected ultrasound parameters could induce membrane poration, enabling transient nanoparticle entry into cells, fluorescent dyes—Hoechst 33342, calcein acetoxymethyl ester (calcein-AM), and propidium iodide (PI)—were used to distinguish viable, dead, and porated cells. PI enters the cytoplasm only when pores are present in the cell membrane, allowing its fluorescence signal to serve as a marker of membrane disruption. Cell viability was assessed using calcein-AM, which is converted by intracellular esterases into the green-fluorescent compound calcein in live cells. Cells expressing green (calcein) and red (PI) fluorescence were thus identified as cells with ultrasound-induced sonoporation effect. The sonoporation rate (%) was calculated as the number of cells exhibiting both green and red fluorescence divided by the total number of cells expressing green and/or red fluorescence. To evaluate whether sonoporation varied with total exposure time and duty cycle, ultrasound conditions representing low, medium, and high energy levels were examined.

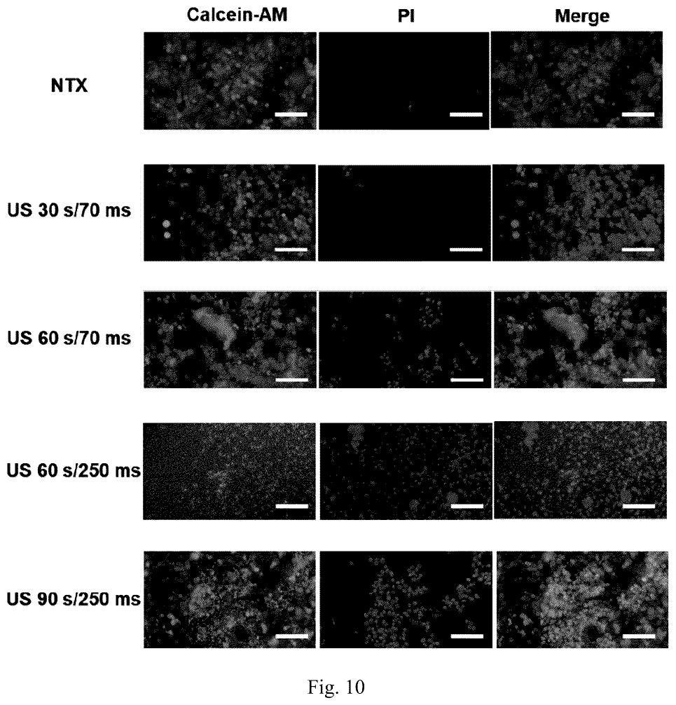

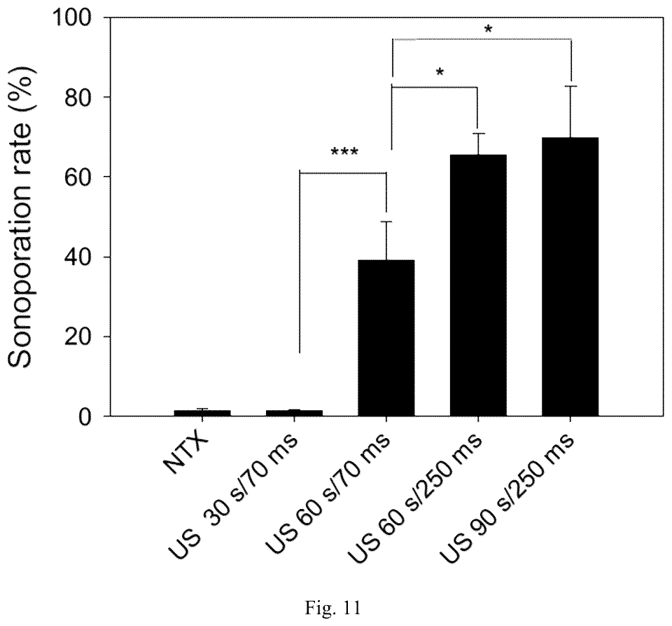

As shown in FIGS. 10 and 11, no significant sonoporated cells were observed in either

the untreated control group (NTX) or the group exposed to ultrasound for 30 seconds at a duty cycle of 70 ms (US 30 s/70 ms), with sonoporation rates of 1.35% and 1.34%, respectively. In contrast, ultrasound exposure for 60 seconds at a duty cycle of 70 ms resulted in a sonoporation rate of 39.47%, while 60 seconds at 250 ms and 90 seconds at 250 ms yielded 65.59% and 69.80%, respectively. These findings demonstrate a significant increase in the proportion of sonoporated cells with increasing ultrasound energy.

### EXAMPLE 10

### Effect of Ultrasound Therapy on Tumor Volume Changes

Experiments were conducted using wild-type C57BL/6 mice aged 6-8 weeks. After a 7-day acclimation period, the mice were anesthetized via inhalation anesthesia, and MOC2 cells (30,000 cells/10 μL of PBS+10 μL of Matrigel) were injected into the mucosa on the inner side of the right lip. Seven days after tumor implantation (day 7), a high-frequency ultrasound imaging system was used to measure and confirm tumor growth by measuring the length, width, and height of the tumor in the axial and longitudinal axes. Treatments were administered on days 8, 10, and 13, followed by ultrasound imaging on days 10 and 14 to evaluate post-treatment tumor growth.

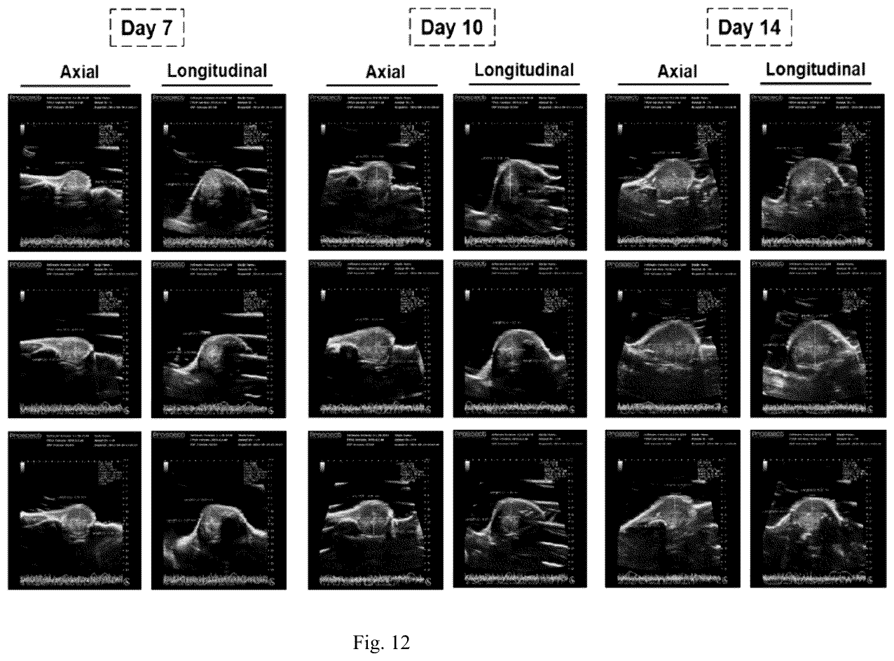

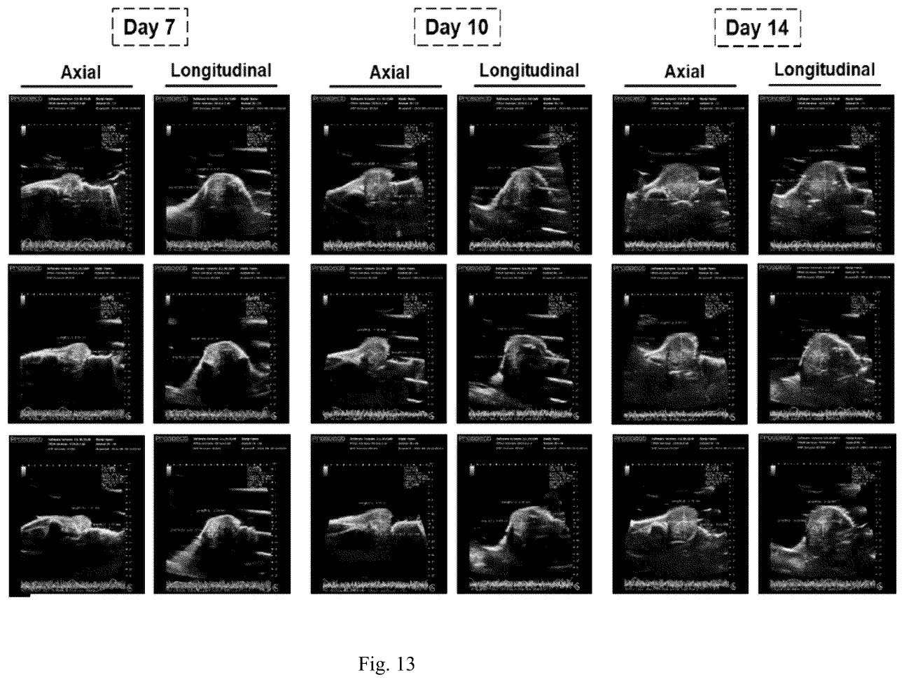

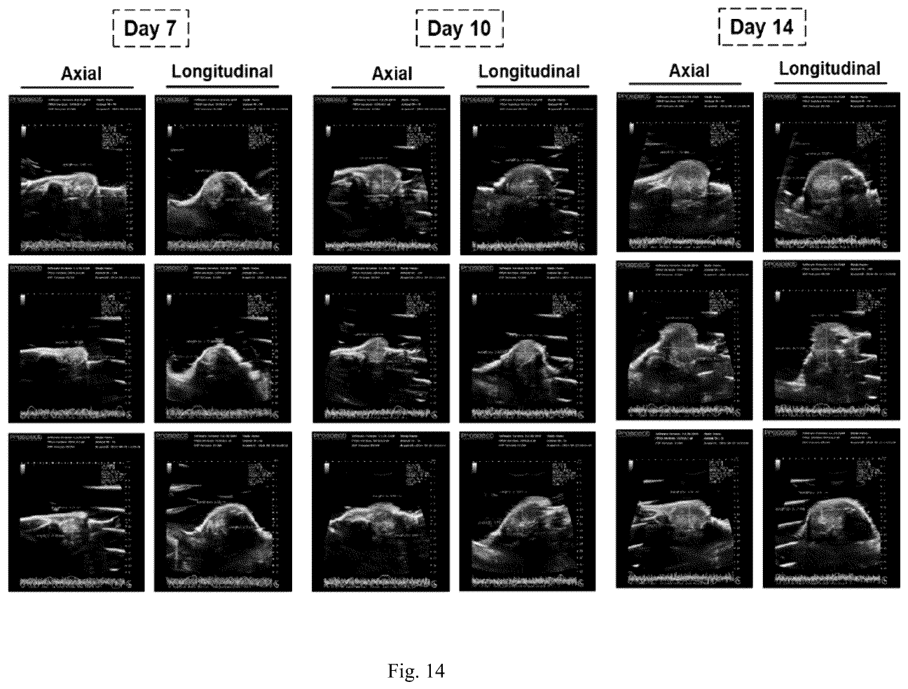

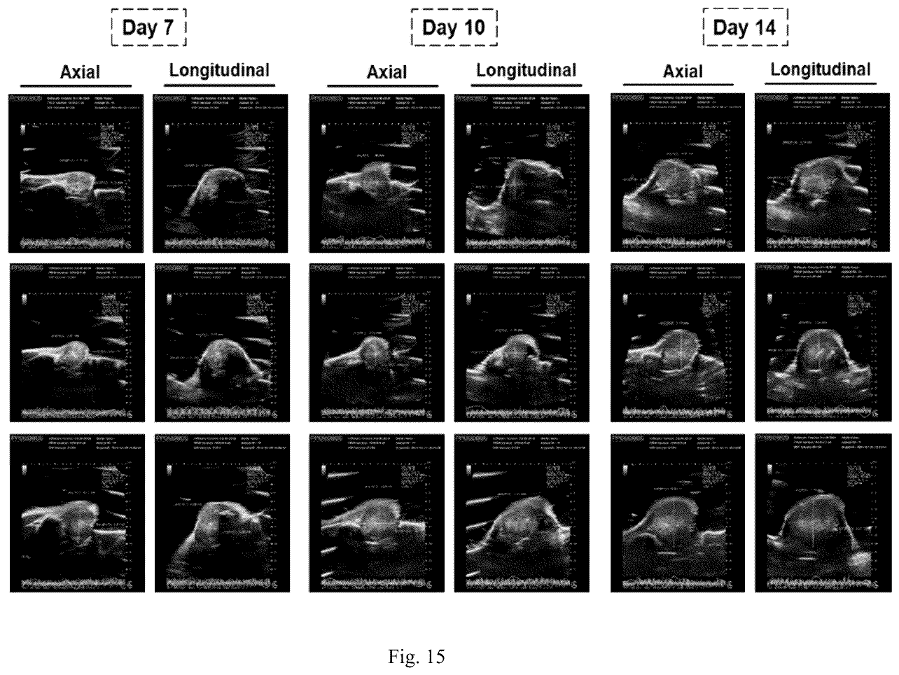

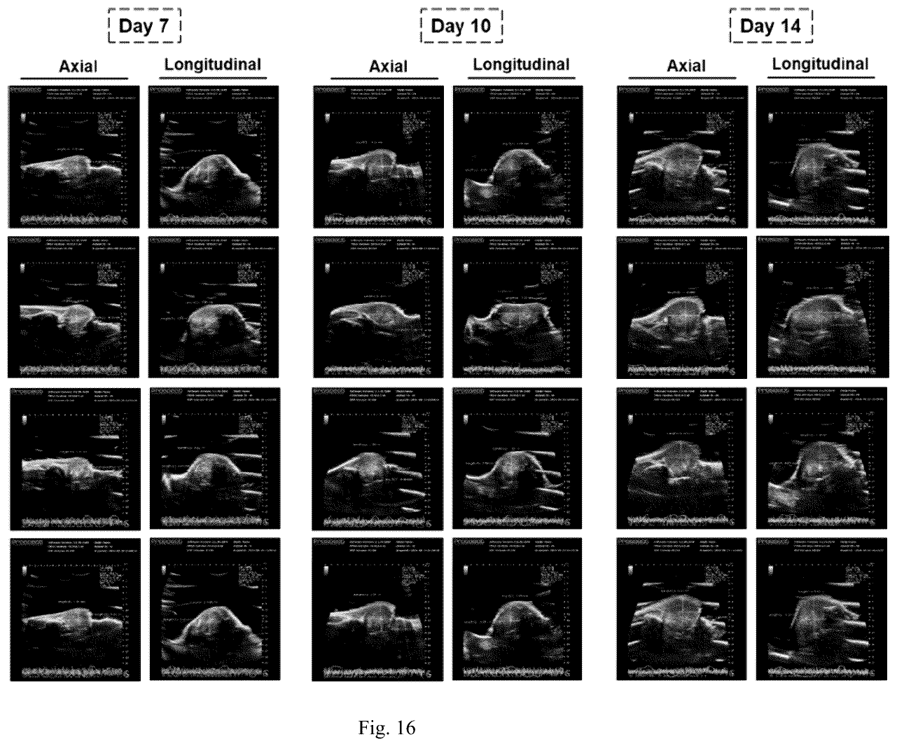

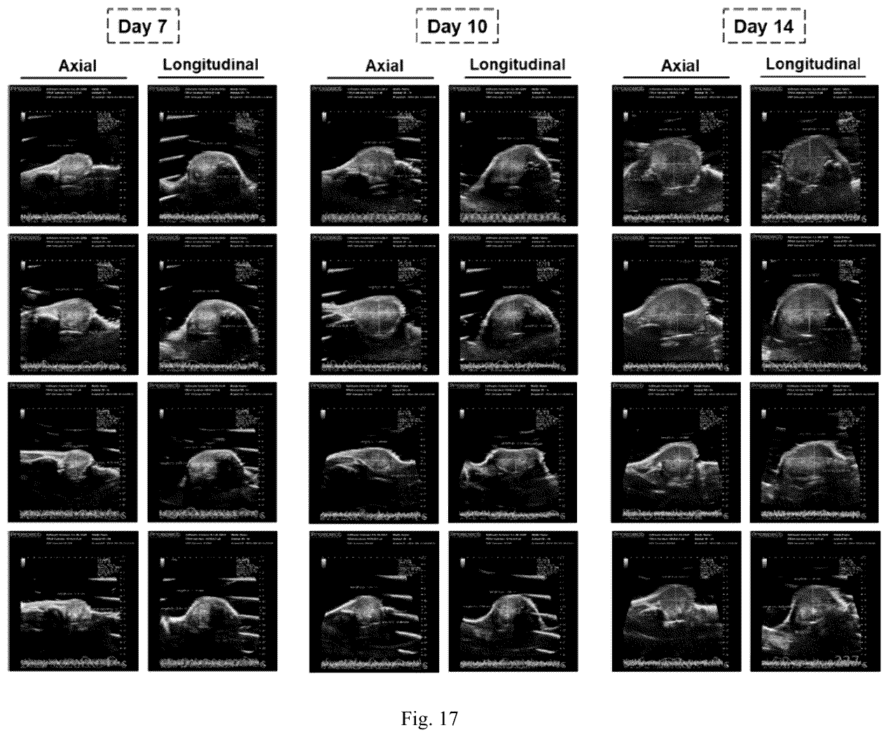

Mice were randomly assigned to six experimental groups (n=4 per group): Group 1, negative control (NTX, FIG. 12); Group 2, agarose patch passive diffusion (aCisNP, FIG. 13); Group 3, ultrasound exposure for 60 seconds and 70 ms duty cycle (US 60 s/70 ms, FIG. 14); Group 4, ultrasound exposure for 60 seconds and 250 ms duty cycle (US 60 s/250 ms, FIG. 15); Group 5, ultrasound exposure for 60 seconds and 70 ms duty cycle with drug patch treatment (US 60 s/70 ms+aCisNP, FIG. 16); and Group 6, ultrasound exposure for 60 seconds and 250 ms duty cycle with drug patch treatment (US 60 s/250 ms+aCisNP, FIG. 17).

Tumor volume was calculated from ultrasound-derived three-axis measurements using the formula V=π/6×a×b², where a is the long axis and b is the short axis. Tumor growth rates were determined by normalizing tumor volume at days 10 and 14 to baseline tumor volume on day 7.

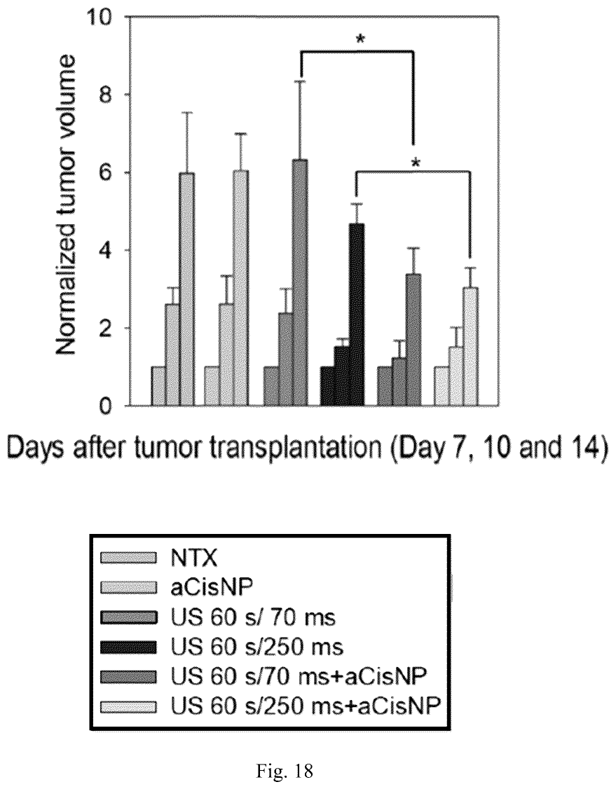

As shown in FIG. 18, tumor growth ratios were calculated using the tumor size calculated on day 7 as the baseline. Tumor growth ratios on day 7, 10, and 14 were as follows: NTX group, 1.00, 2.61, and 5.98; aCisNP group, 1.00, 2.62, and 6.03; US 60 s/70 ms group, 1.00, 2.38, and 6.33; US 60 s/250 ms group, 1.00, 1.53, and 4.69; US 60 s/70 ms+aCisNP group, 1.00, 1.23, and 3.39; and US 60 s/250 ms+aCisNP group, 1.00, 1.52, and 3.03.

The results indicated no significant difference between the agarose patch passive diffusion group (aCisNP) and the negative control group (NTX), demonstrating that passive drug release without external facilitation failed to inhibit tumor growth significantly. In contrast, within the ultrasound-treated groups, the US 60 s/70 ms+aCisNP group showed significant differences compared with both NTX and US 60 s/70 ms alone, while the US 60 s/250 ms+aCisNP group exhibited significantly reduced tumor growth compared with both NTX and US 60 s/250 ms. These findings indicate that both ultrasound parameter sets effectively facilitated chemotherapeutic drug delivery and suppressed in vivo tumor growth.

### EXAMPLE 11

### Determination of Platinum (Pt) Content in Tumor Tissues

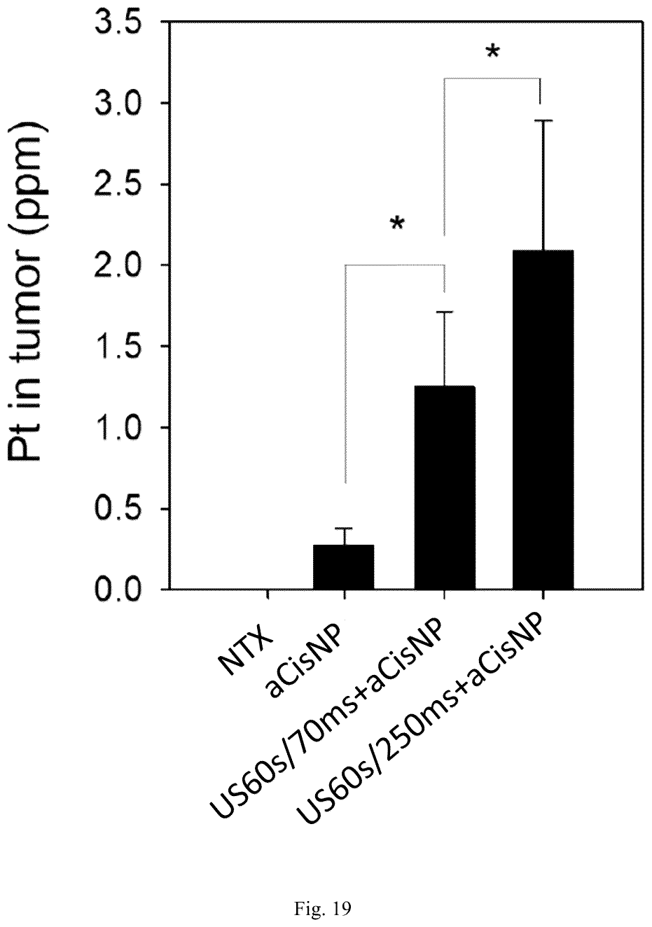

To determine whether cisplatin penetrated the tumor tissue, elemental analysis was performed using inductively coupled plasma mass spectrometry (ICP-MS). Excised tumors were frozen, subjected to microwave digestion, and analyzed for Pt content. As shown in FIG. 19, no detectable Pt content was observed in the untreated control group (NTX), while a trace amount (0.27 ppm) was detected in the passive release group (aCisNP). In contrast, Pt content was substantially higher in the ultrasound-assisted delivery groups, with 1.24 ppm detected in the US 60 s/70 ms+aCisNP group and 2.08 ppm in the US 60 s/250 ms+aCisNP group. These findings are consistent with previous experimental results, demonstrating that ultrasound-assisted drug delivery (US 60 s/70 ms and US 60 s/250 ms) enhances intratumoral cisplatin accumulation.

### CONCLUSION

This study demonstrated that ultrasound-mediated physical forces enhance the delivery of CisNP by increasing skin permeability in mice, with higher ultrasound energy inducing greater drug release rates. Elevated sonoporation rates were observed in groups with enhanced cisplatin release, indicating that non-bubble-based sonoporation may serve as a potential underlying mechanism of this ultrasound-assisted drug delivery platform. Similarly, in vivo experiments confirmed the tumor-inhibitory effect of ultrasound-facilitated drug delivery through analysis of tumor volume changes. Furthermore, ICP-MS analysis verified cisplatin accumulation within the tumors, confirming successful drug delivery to the tumor site.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

What is claimed is:

1. An ultrasound-assisted drug-loaded chitosan nanoparticles delivery platform comprising:
supplying drug-loaded chitosan nanoparticles;
providing an ultrasound stimulation;
administering the drug-loaded chitosan nanoparticle to a target region; and
applying ultrasound mechanical force to the target region,
and wherein the ultrasound stimulation induces localized deformation of cell membranes, resulting in transient increases in membrane permeability that enable the intracellular delivery of the drug.

2. The ultrasound-assisted drug-loaded chitosan nanoparticles delivery platform of claim 1, wherein the ultrasound stimulation applies to the target region via a low-frequency non-focused or focused probe.

3. The ultrasound-assisted drug-loaded chitosan nanoparticles delivery platform of claim 1, wherein the target region is oral.

4. The ultrasound-assisted drug-loaded chitosan nanoparticles delivery platform of claim 2, wherein the operating time provided by the low-frequency non-focused or focused probe is at a duty cycle of 50% and within a range of 50 to 300 ms.

5. The ultrasound-assisted drug-loaded chitosan nanoparticles delivery platform of claim 2, wherein the ultrasonic stimulation of a low-frequency non-focused or focused probe is between 50 and 90 seconds.

6. The ultrasound-assisted drug-loaded chitosan nanoparticles delivery platform of claim 1, wherein the ultrasonic stimulation of a low-frequency non-focused or focused probe is 60 seconds.

7. The ultrasound-assisted drug-loaded chitosan nanoparticles delivery platform of claim 1, wherein the drug-loaded chitosan nanoparticles are loaded with cisplatin molecules.

8. The ultrasound-assisted chitosan nanoparticle drug delivery platform of claim 1, wherein the drug-loaded chitosan nanoparticles are further provided in a drug patch.

9. The ultrasound-assisted drug-loaded chitosan nanoparticles delivery platform of claim 1, wherein the particle size of the drug-loaded chitosan nanoparticle is 300-500 nm.

10. The ultrasound-assisted drug-loaded chitosan nanoparticles delivery platform of claim 1, wherein the particle size of the drug-loaded chitosan nanoparticle is 300-400 nm.
